(19) United States
(12) Patent Application Publication
(10) Pub. No.: US 2007/0105822 A1
(43) Pub. Date: May 10, 2007

(54) Novel uses of known drugs

(76) Inventor: Andrew Sewell, Oxford (GB)

Correspondence Address: BELL & ASSOCIATES, 416 FUNSTON ST., SUITE 100, SAN FRANCISCO, CA, 94118, US

(21) Appl. No.: 10/580,658

(22) PCT Filed: Nov. 29, 2004
(86) PCT No.: PCT/GB04/05019

§ 371 Date: May 26, 2006

(30) Foreign Application Priority Data
Nov. 28, 2003 (GB) 0327742.3

(51) Int. Cl. A61K 31/663; A61K 31/401; A61K 31/675; A61K 31/366; A61K 31/22

(52) U.S. Cl. 514/89; 514/102; 514/423; 514/460; 514/548

(57) ABSTRACT

Statins are able to block the mevalonate pathway at an early stage, preventing build up of isopentenyl pyrophosphate by nitrogen containing bisphosphonates by blocking the pathway further downstream, with medicaments containing statins being administered at an appropriate time being able to thereby prevent the build up of IPP, which has been found to activate γδ T cells and cause the acute phase response associated with the nitrogen containing bisphosphonates.

### FIELD OF THE INVENTION

The present invention relates to the use of statins, and other early mevalonate pathway inhibitors, in medicine.

### BACKGROUND OF THE INVENTION

The bisphosphonates are useful in various clinical applications worldwide (1), particularly in the treatment of musculoskeletal disorders, and they are now amongst the most commonly used agents in clinical practice. They have proven to be particularly useful in the management of osteoporosis, but also in other disorders of the bone and joint, such as Paget's disease, skeletal metastases, fibrous dysplasia, Charcot's arthropathy, sympathetic dystrophy, pachydermic periostosis and aseptic osteomyelitis.

Bisphosphonates (BPs) are commonly used for the treatment of Paget's disease, multiple myeloma, osteoporosis and hypercalcaemia (reviewed in (1)). The clinical success of the BPs etidronate and clodronate, in the 1970s and 1980s, led to trials of BPs with different alkyl chains. In particular, bisphosphonates containing an amino group in their alkyl chain, such as pamidronate and alendronate, were found to be 10- to 100-fold more potent at inhibiting bone resorption than the early non-amino bisphosphonates (15). Risedronate and zoledronate contain a nitrogen atom within a heterocyclic ring (16) and have been shown to be up to 10,000-fold more potent than etidronate, in experimental systems (17).

Mechanistic studies indicate that the bisphosphonates can, broadly speaking, be classified into two groups, based on their mode of action: those that resemble pyrophosphate, including etidronate, clodronate, and tiludronate, and which can be incorporated into cytotoxic ATP analogues; and the more potent nitrogen-containing bisphosphonates that interfere with other metabolic pathways, such as the mevalonate pathway (reviewed in (1)).

Inhibition of the mevalonate pathway by nBPs leads to an accumulation of intermediates, including isopentenyl pyrophosphate (IPP), in this pathway (18). IPP is a potent activator of human peripheral blood γδT cells (19) and nBPs have also been described as activating these cells (18, 20-23). The acute phase response has not been observed with the non-aminobisphosphonates, etidronate, clodronate or tiludronate, and appears to be a specific feature of the nBPs (1).

The dominant subset of human γδ T cells in peripheral blood bear T cell receptors (TCRs) encoded by the Vγ9 and Vδ2 genes. These cells are known to activate in response to alkylphosphate, alkylamine and some bisphosphonate antigens. This recognition is a characteristic of cells bearing a Vγ9 chain that uses the JP joining region (24). Vγ9JP-expressing cells are highly enriched in the peripheral blood, compared to percentages in thymocytes or cord blood (24, 25), which is suggestive of antigen-induced expansion.

Bisphosphonates, and in particular the aminobisphosphonates (nitrogen-containing bisphosphonates, or nBPs), are known to have a number of side effects (reviewed in (1)) including a rise in body temperature and accompanying flu-like symptoms which resemble a typical acute phase response (2, 3), and was first described over 15 years ago. The mechanism for this response has been partially elucidated and appears to be associated with the release of tumour necrosis factor alpha (TNFα) and IL6 (4-6), although the effector cells that release these cytokines and the mechanism of action remain unknown.

The cellular mechanism for the action of nBPs leading to production of TNFα and IL6 has remained unclear, although monocytes and/or macrophages have been mooted as likely candidates for releasing these pro-inflammatory agents (5). In addition, there has been considerable concern over gastrointestinal disturbances associated with oral administration of the nBP alendronate (28). Similar problems had earlier led to the discontinuation of oral pamidronate for osteoporosis (29), although administration of these compounds by intravenous or intramuscular injection is increasing in popularity worldwide, as these routes avoid gastrointestinal disturbances. It would clearly be advantageous to find a way to be able to administer nBPs orally, once again, without the above-identified problems.

Bukowski et al. (31) demonstrated that a single Vγ9Vδ2 T cell clone could recognise two types of ligand, alkylamines and IPP, and suggested that, given the small sizes of these antigens, a single γδ TCR might possess two different binding sites. The similarity between the recognised ligands is their alkyl chains. It is known that phosphate antigens require their phosphate, and nBPs and alkylamines require their amino groups, for their antigenicity (1, 19, 20). Therefore, it has been assumed that these groups form a component of the TCR-docking structure. The finding that both antigenic alkylphosphate and alkylamine ligands require similar alkyl chains for recognition strengthened this assumption, and lead to attempts at molecular prediction (27).

The most potent nBP antigen to date, risedronate, which can stimulate γδ T cell expansion from human PBMC at concentrations as low as 10 nM (21), was not included in this predictive exercise (27). Risedronate has an aromatic ring and does not fit with the pattern recognition hypothesis championed by Gossman and Oldfield (27).

US 2001/0036936 (Pfizer) discloses treatments for atherosclerosis and for promoting bone growth, comprising polyphosphonates in combination with statins, the former to prevent bone resorption and the latter as bone forming agents. There is no discussion of any potential inflammatory effects of bisphosphonate.

U.S. Pat. No. 6,080,779 discloses a combination of statins together with other compounds, such as oestrogens and/or bisphosphonates and/or bone growth-promoting compounds to treat various bone conditions, such as osteoporosis.

WO 02/011704 discloses diphosphonates, optionally administered with a statin, to promote bone growth.

WO 01/46206 discloses phosphonic acid derivatives as being useful in the treatment of PTP-1P related conditions, such as diabetes, and mentions that statins can be used, in their capacity as cholesterol biosynthesis inhibitors.

WO 99/45923 discloses statins, which can be used in combination with bisphosphonates, to inhibit bone resorption.

US 2001/0025028 discloses statins, with optional bisphosphonates, also to inhibit bone resorption.

Surprisingly, it has now been established that the nBPs are not acting as an antigen at all, which helps to explain why risedronate does not fit the pattern recognition hypothesis, but are acting via their effect on the mevalonate pathway. Thus, rather than acting directly on monocytes and/or macrophages, it is their ability to raise levels of intermediary metabolites found in the mevalonate pathway that stimulates the acute phase effect and, to this extent, it has also been found that the effect is mediated by the γδ T cells. This effect can be ameliorated or blocked by blocking the mevalonate pathway at a stage before the activating intermediates are produced.

### SUMMARY OF THE INVENTION

Thus, in a first aspect, the present invention provides a pharmaceutical formulation comprising an early-stage mevalonate pathway blocker and a bisphosphonate. An early-stage mevalonate pathway blocker is one that blocks the mevalonate pathway at a juncture before the production of IPP, preferably two or more steps before, generally either by inhibiting or blocking an enzyme, chelating a reagent, preventing production of an enzyme, or by some other suitable means.

In an alternative aspect, the present invention provides the use of an early stage mevalonate pathway blocker in the manufacture of a medicament for the prevention or amelioration of an acute phase response in a patient being treated or about to be, or intended to be, treated with a bisphosphonate. In this respect, a patient about to be treated is one that it is intended to receive bisphosphonate, during a period in which the early-stage mevalonate pathway blocker is still effective to block or substantially restrict the mevalonate pathway in the patient after administration. It is then preferable to continue treatment with the statin for the duration of treatment with the bisphosphonate, or at one or more intervals prior to each administration of bisphosphonate, as discussed below. The bisphosphonate will preferably be one that has been, or is, associated with an acute phase response.

There is also provided a method for preventing or ameliorating an acute phase response in a patient receiving, or about to receive, bisphosphonate treatment, comprising administering an effective amount of an early-stage mevalonate pathway blocker thereto. The patient may be human or non-human, such as a horse or cat, for example. It is preferred that the patient be human.

In a further aspect, the present invention provides the use of an early-stage mevalonate pathway blocker and a bisphosphonate in the manufacture of a medicament for the treatment or prophylaxis of a condition treatable by the bisphosphonate, the early-stage mevalonate pathway blocker being present in an amount effective to block an early stage of the mevalonate pathway.

In a yet further aspect, there is provided the use of an early-stage mevalonate pathway blocker in the manufacture of a medicament for the treatment or prophylaxis of side effects associated with a bisphosphonate.

There is also provided a kit comprising a first medicament comprising an early-stage mevalonate pathway blocker and a second medicament comprising bisphosphonate, preferably together with an indication that the first medicament should be taken no later than the second.

### DESCRIPTION OF THE DRAWINGS

The invention will now be described with reference to the accompanying Figures in which:

FIG. 1: nBPs induce rapid and copious production of TNFα by peripheral blood γδ T cells;

In FIG. 1 (A) nBPs, but not non-aminoBPs, are shown to activate a Vγ9JPVδ2 cell clone Bob in IFNγ ELISpot.

In FIG. 1 (B), nBPs are shown to stimulate TNFα production from direct ex vivo human PBMC.

In FIG. 1 (C), activation of Vγ9JPVδ2 T cell clones by risedronate is shown to be extremely rapid.

FIG. 2 shows how the TNFα and IL6 produced by PBMC in response to nBPs is derived from γδ T cells.

Magnetic depletion of γδ T cells from human PBMC removes their ability to manufacture TNFα in response to pamidronate (FIG. 2 A) and risedronate (FIG. 2 B).

In FIG. 2 (C), exposure to nBPs activates PBMC that express a Vγ9 receptor and not other T cells or B cells.

In (D), intracellular cytokine staining shows that nBPs induce direct ex vivo Vγ9-expressing T cells to make TNFα and IL6.

FIG. 3 shows that statins inhibit nBP-induced TNFα production by human PBMC.

FIG. 3 (A) shows the results of pre-treatment of human PBMC, where pravastatin, simvastatin, or fluvastatin, inhibits their ability to manufacture TNFα in response to risedronate.

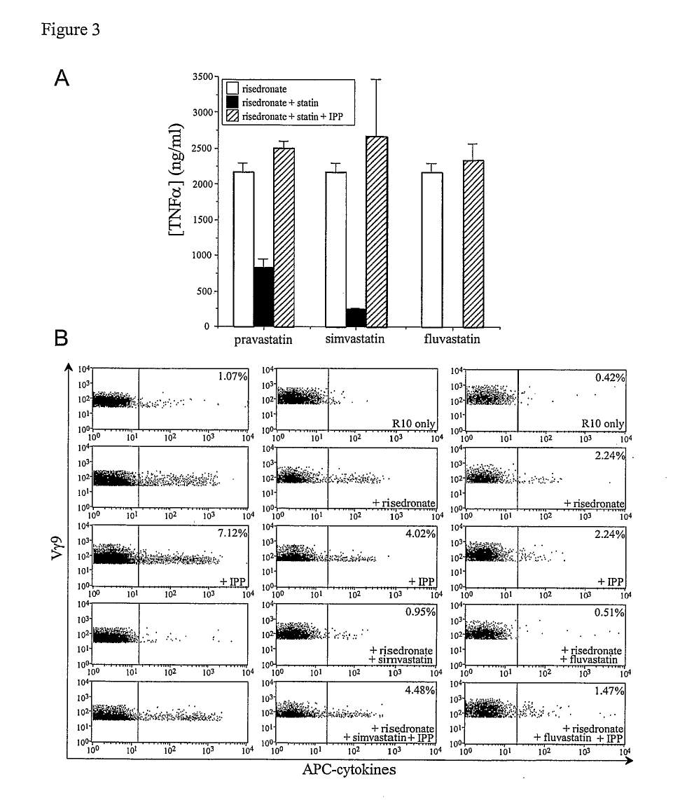

In FIG. 3 (B), it is shown that pre-treatment of PBMC with statins inhibits nBP-induced activation of Vγ9-expressing T cells, by ICS.

### DETAILED DESCRIPTION OF THE INVENTION

The gastrointestinal tract is the main reservoir of γδ T cells in the body, as it is the main portal of entry for bacterial pathogens. Thus, oral administration of the medicaments of the present invention serves to provide protection against the effect of orally administered nBPs, although it will be appreciated that circulatory γδ T cells are generally considered to be those that provide the acute phase response. Indeed, the acute phase response is still observed with intravenous administration of nBPs.

It will be appreciated that any suitable substance suitable for therapeutic administration may be used as an early-stage blocker of the mevalonate pathway. In this respect, “early-stage” indicates that the pathway should be blocked at a stage before any significant amount of γδ activating intermediates is produced. In particular, it has been established that IPP activates γδ T cells, so that it is preferred to block the pathway at a point before the production of IPP. It is more preferred to block at an earlier stage, in order to minimise the possibility of a build up of an earlier intermediate forcing the equilibrium toward IPP.

Suitable substances for use in the present invention to block the mevalonate pathway at an early stage are those substances identified as the statins. The statins are inhibitors of 3-hydroxy-3-methylglutarylcoenzyme A reductase (HMG-CoA reductase), an enzyme which catalyses the conversion of HMG-CoA to mevalonate, which is a required building block for cholesterol biosynthesis. The statins do not all share a similar structure. For example, simvastatin and lovastatin vary by only one methyl, while there is little similarity with atorvastatin or fluvastatin. Likewise, mevinolin shares some similarity with lovastatin, but has a substantially different side-chain. Nevertheless, the skilled person is familiar with the statins, and is readily able to select a statin suitable for administration with the bisphosphonate. Any one of the statins capable of blocking the mevalonate pathway at an early stage may be used alone, or together with any one or more other statins.

Where statins are referred to herein, it will be appreciated that other early-stage mevalonate pathway blockers are also envisaged, unless otherwise apparent or indicated.

In general, the bisphosphonates, whose effect it is desired to counteract, will be the nBPs, and this group is particularly preferred, especially those recognised for their clinical utility. Preferred nBPs include pamidronate, dimethyl pamidronate, and alendronate, which contain a basic primary nitrogen atom in an alkyl chain, and ibandronate, risedronate and zoledronate, which contain a tertiary nitrogen. Any one of the nBPs may be used alone, or together with any one or more other nBPs.

Although the statins may be administered together with the nBPs, it will be appreciated that greater efficiency at blocking the pathway will be achieved by earlier administration. In particular, as the pathway is generally constitutive, blocking it at the beginning will not have an effect downstream for some time, so that a statin co-administered with an nBP will prevent further HMG-QoA entering the pathway, but that which is already on the pathway can still be blocked by the nBP, leading to an accumulation of IPP. However, the overall quantities will be less, and it may often be sufficient to co-administer the statin and nBP.

However, it is preferred to administer the statin from several hours before the nBP to several days before. Where the product is a kit, for example, it is preferred to indicate this on the pack, and to provide extra medicament comprising statin, for the purpose.

It will also be appreciated that, after the initial statin application or course, it will generally be acceptable to co-administer the statin with the nBP for the remainder of the treatment.

Preferred conditions treatable by the compositions of the present invention include any treatable by a bisphosphonate which is known to be, or suspected of being, associated with an increased risk of an acute phase response. As the statins are capable of blocking the pathway leading to the production of IPP, which appears to be responsible for, or at least instrumental in, any observed acute phase response, then it is now possible to administer bisphosphonates with a reduced or eliminated risk of causing such a response. Thus, it is now possible to administer bisphosphonates orally, with little or no risk of this leading to an acute phase response, the contraindications having been completely, or largely, circumvented.

Preferred conditions are musculoskeletal disorders, including osteoporosis, Paget's disease, skeletal metastases, fibrous dysplasia, Charcot's arthropathy, sympathetic dystrophy, pachydermic periostosis, aseptic osteomyelitis, multiple myeloma, hypercalcaemia, atherosclerosis and PTP-1P related conditions, such as diabetes.

It is a particular advantage of the present invention that it is now possible to administer nBPs orally with substantially reduced likelihood of acute phase side effects. Thus, oral preparations are preferred, and include such formulations as tablets, capsules, elixirs and syrups, and may contain suitable excipients and flavourings. Tablets may be formulated simply with a carrier or may be enterically coated, for example.

Any formulation of bisphosphonate or statin is acceptable, especially where conventionally formulated, and irrespective of whether the two are formulated together or separately. Both may be formulated for oral administration and, especially after the beginning of the course, an oral formulation may comprise both actives. However, either, or both, may be formulated for intravenous, intramuscular or any other injectable format, or may be formulated as creams, ointments, gels, lotions, eye drops, pessaries, suppositories, patches or subdermal matrices, for example, if desired.

Suitable quantities of statins and nBPs for use in the invention will be readily discerned by those skilled in the art, and will generally correspond to those known to be effective for the, or other, indications of the compounds. It is generally desirable to use the same or higher concentrations of the statins, at least to start, and preferably for the duration, than are used to control levels of cholesterol in a patient. However, the primary requirement is that the IPP production pathway be inhibited or, more preferably, blocked, so that all that is required is for sufficient levels of statin to be administered to achieve this, whether continually, such as via a patch or drip, or intermittently, such as by tablet or capsule. In the case of a capsule, particles of the statin may be enterically coated, for example, to provide a sustained release effect.

The anti-inflammatory effects of the statins appear to be had by inhibiting the activation of human peripheral blood γδ T cells. The activation is caused by way of response to endogenous non-sterol mevalonate products whose presence is subtsantially diminished after treatment with a statin. The effect of statins on γδ T cells is particularly advantageous in the treatment of autoinimune conditions, and especially those where inflammation has been identified as being a contributory element in the disease process, such as reactive arthritis and multiple sclerosis, where the γδ cells have been implicated as being causal.

Thus, in a yet further aspect, the present invention provides the use of statins in the manufacture of a medicament for the treatment of inflammation in an autoimmune disease. The autoimmune disease is preferably one in which γδ cells have been identified as being a causative agent, and is especially multiple sclerosis or reactive arthritis.

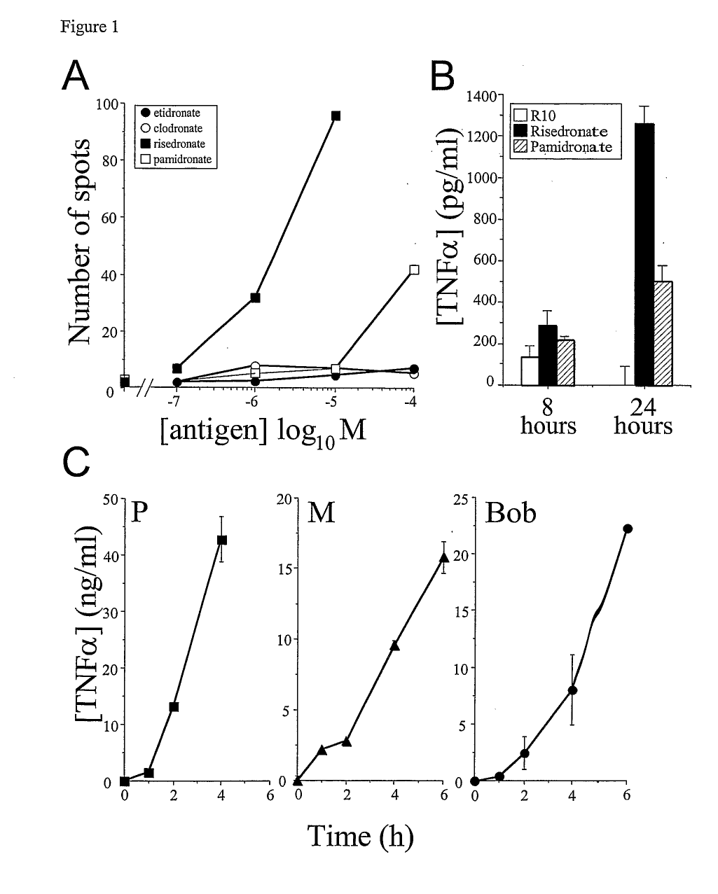

As verification of the present invention, a clonal population of Vγ9JPVδ2 T cells was shown to activate after exposure to the nBPs, pamidronate and risedronate, but not the nonaminoBPs, etidronate and clodronate, by INFγELISpot (FIG. 1A). Vγ9JPVδ2 T cells also made large amounts of TNFα after exposure to nBPs (FIG. 1C). Production of TNFα was both copious and rapid (FIG. 1C). A Vγ9JPVδ1-expressing clone was unable to recognise these ligands, indicating that the δ2 chain also plays an essential role in the response of Vγ9JPVδ2 T cells to nBPs.

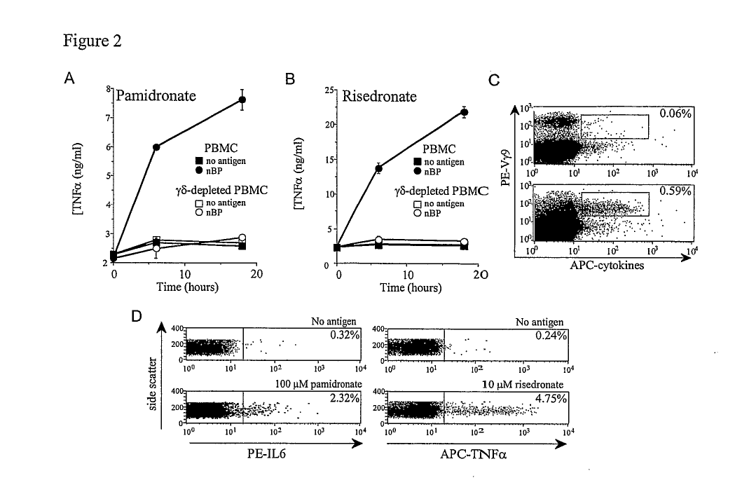

Pamidronate and risedronate were also able to induce large amounts of TNFα from direct ex vivo human peripheral blood mononuclear cells (PBMC) (FIG. 1B). Production of this cytokine was rapid (FIG. 1B). Magnetic depletion of γδ T cells removed the ability of PBMC to produce TNFα in response to pamidronate (FIG. 2A) and risedronate (FIG. 2B). Intracellular cytokine staining (ICS) showed that exposure of PBMC to nBPs activated the peripheral blood lymphocyte population bearing a Vγ9 TCR and not other T cells or B cells (FIG. 2C). Vγ9-expressing cells were shown to make both IL6 and TNFα in response to pamidronate and risedronate by ICS (partially shown in FIG. 2D).

A previous study (26) found that 4 in 10 patients given 60 or 90 mg infusions of pamidronate had an acute phase reaction. All of these patients showed a substantial increase in the number of circulating γδ T cells when measured 1 and 3 weeks post-infusion (26). In one patient γδ T cells expanded from 4.6% to 70% of CD3+ cells post-infusion.

In order to demonstrate that nBPs exert their effect on the activation of Vγ9JPVδ2 T cells via the mevalonate pathway, and not to act as direct ligands for the γδ TCR as had been thought in previous studies (20-23, 27), inhibitors of HMG CoA were shown to be able to inhibit the nBP-induced activation of γδ T cells directly ex vivo (FIG. 3).

Pravastain, simvastatin and fluvastatin were all able to inhibit TNFα production by fresh PBMC, as measured by ELISA (FIG. 3A). Addition of IPP was able to rescue TNFα production, ruling out the possible toxic affects of these statins at the concentrations used. These statins were also shown to inhibit the activation of Vγ9 cells by ICS (FIG. 3B). Once again, addition of IPP in addition to the statin was able to rescue activation and rule out toxic effects.

It has now been shown that nBPs induce rapid and copious production of the pro-inflammatory cytokines by human peripheral γδ T cells (FIGS. 1-3), with depletion of γδ T cells from PBMC abrogating this effect (FIG. 2), and that statins, which inhibit HMG CoA reductase, are able to inhibit nBP-induced activation of γδ T cells (FIG. 3).

It is further noteworthy that statins have anti-inflammatory properties (7-14) and appear to promote a Th2 cytokine bias in vivo (30); the therapeutic relevance of these pharmacologic effects is becoming increasingly apparent. The mechanism of such effects is still under debate. Our work indicates that some of the anti-inflammatory effects of these compounds could potentially result from their ability to inhibit the activation of γδ T cells, a result that could shed light on the physiological relevance of these lymphocytes in immune regulation.

The present invention will now be illustrated by the following, non-limiting Example.

### EXAMPLE

In the following Example, the materials and methods were as follows:

Drugs

The following bisphosphonates and statins were used: disodium etidronate (Procter & Gamble Pharm), disodium clodronate (Roche), disodium pamidronate (Novartis), risedronate sodium (Procter & Gamble Pharm), simvastatin (Ranbaxy Laboratories Limited), pravastatin (Bristol-Myers Squibb Pharmaceuticals) and fluvastatin (Sandoz Pharmaceuticals).

γδ T Cell Culture

γδ T cells were isolated from human peripheral blood using a magnetic separation kit (TCR γ/δ Microbead Kit and MS columns from Miltenyi Biotech). Once isolated, cells were resuspended in T cell medium (RPMI supplemented with 100 U/ml penicillin, 100 μg/ml streptomycin, 10% FCS, 10% T-STIM (BD Biosciences), 200 U/ml Proleukin (Chiron)) containing 2×10⁶ γ-irradiated PMBC/ml from three individuals and 2 μg/ml PHA. Cells were maintained with T cell medium, and restimulated with mixed irradiated PBMC and PHA every three weeks. For cloning, the same mix was used to grow cells by limiting dilution in 96-well U-boftomed plates. Cells that grew were confirmed to be γδ T cells with Vγ9 antibody.

Antibodies

The following antibodies were used for flow cytometric analysis: FITC-conjugated mouse anti-human Vδ2 TCR mAb clone B6.1 (PharMingen), FITC-conjugated mouse anti-human Vγ9 mAb clone 7A5 (Endogen), FITC-conjugated mouse anti-human Vδ1 mAb clone TS8.2 (Endogen), PE-conjugated mouse anti-human Vγ9 mAb clone B3.1 (PharMingen), FITC-conjugated mouse anti-human pan γδ mAb clone 5A6.E9 (Endogen), PerCP-conjugated mouse anti-human CD3 mAb clone SK7 (BD Biosciences), allophycocyanin (APC)-conjugated anti-human IL2 clone MQ1-17H12 (Caltag), APC-conjugated anti-human TNFα clone mAb11 (Pharmingen), APC-conjugated anti-human IFNγ clone B27 (Pharmingen) and PE-conjugated anti-human IL6 clone AS12 (BD Biosciences).

γ-IFN ELISpot and TNF α ELISA

γδ T cells were washed in RPMI and incubated overnight in R10 at 37° C. 96-well nitrocellulose plates (Millipore) were incubated overnight at 4° C. with 15 μg/ml antihuman-IFN-γ primary antibody (clone 1-D1K; Mabtech, Sweden). The plates were then washed twice with RPMI and blocked with R10 for 3 hours at 37° C. R10 was decanted by inversion and assays applied to each well before incubation at 37° C. as detailed below. Assays were terminated by washing once in water, followed by 5 washes in PBS. Secondary antibody (anti-human-IFN-γ-Biotin antibody clone 7-B6-1; Mabtech) was applied at 1 μg/ml and the plate incubated for 100 minutes at room temperature (RT). The plate was washed 6 times with PBS before application of Streptavidin-ALP (1:1000 in PBS; Mabtech) for 40 minutes at RT. After 6 further washes in PBS, spots were revealed by incubation for 15 minutes at RT with developing buffer (Bio-Rad AP conjugate substrate kit) and counted mechanically using an ELISpot Reader System ELR02 (Autoimmun Diagnostika; Strassberg). A TNFα ELISA kit (Peprotech) was used in accordance with the manufacturers instructions.

Intracellular Cytokine Staining

10⁶ fresh PBMC were incubated in FACS tubes with brefeldin A (10 μg/ml R10) for 5 hours after pulsing with relevant antigens for 1 hour. The cells were then washed, permeabilised in 20% FACSLyse (BD Biosciences), washed twice in ice cold PBS/0.1% BSA and stained on ice with pre-titred APC-conjugated anti-IFNγ, anti-TNFα and anti-IL-2 and PE-conjugated anti-Vγ9 mAbs for 20 minutes. For single cytokine analysis (FIG. 2D), cells were stained with PE-conjugated anti-IL6, APC-conjugated anti-TNFα and FITC-conjugated anti-Vγ9 antibodies. In each case, cells were then washed, resuspended in PBS & analysed immediately.

γδ T Cell Depletion of PBMC

10⁸ human PBMC were incubated with 10 μg/ml of anti-human pan-γδ antibody for 30 minutes on ice. Cells were washed once in 20× labelling volume with PBS/0.1%BSA and resuspended at 4×10⁷ cells/ml in PBS/0.1%BSA. 2×10⁷ anti-mouse IgG1 Dynabeads (Dynal) washed in PBS/0.1% BSA were added and the cells incubated for 1 hr at 4° C. with gentle rotation. The tube was then placed in the magnetic particle concentrator (Dynal) and left to separate for 30 minutes. The supernatant was transferred to a fresh tube. The success of depletion was determined by FACS analysis with the anti-CD3 and pan-γδ antibodies detailed above, and was >95% efficient in all cases shown.

The results are shown in the accompanying Figures.

FIG. 1 shows that nBPs induce rapid and copious production of TNFα by peripheral blood γδ T cells.

In FIG. 1 (A) nBPs, but not non-aminoBPs, are shown to activate a Vγ9JPVδ2 cell clone Bob in IFNγ ELISpot. ELISpots were performed with 1,000 Vγ9JPVδ2 T cells and 25,000 spinner HeLa cells as antigen presenting cells per well and incubated for 6 hours prior to development. Standard error from the mean of two replicate assays is shown although, in all cases, these errors are smaller than the plot symbol.

In FIG. 1 (B), nBPs are shown to stimulate TNFA production from direct ex vivo human PBMC. 10⁶ fresh human PBMC were incubated for 12 hours in 75×5 mm FACS tubes at 37° C. and 5% CO₂ in 1 ml of R10 (RPMI, 10% FCS, 100 u/ml penicillin, 100 μg/ml streptomycin), 1 ml of R10+10 μM risedronate or 1 ml of R10+100 μM pamidronate. 60 μl aliquots were removed at the specified time and added to TNFα ELISA assays.

In FIG. 1 (C), activation of Vγ9JPVδ2 T cell clones by risedronate is shown to be extremely rapid. The standard deviation from the mean of two replicate TNFα assays is shown for three separate clones expressing a Vγ9JPVδ2 TCR. Clone P is CD8α+, Clones M and Bob are CD8−. Clone P appears to make more TNFα and to produce it earlier. 10⁶ T cells were activated by 10 μM risedronate in 1 ml of media in 75×5 mm FACS tube at 37° C. and 5% CO₂. 60 μl aliquots were removed at the specified time and added to TNFα ELISA assays.

FIG. 2 shows how the TNFα and IL6 produced by PBMC in response to nBPs is derived from γδ T cells.

Magnetic depletion of γδ T cells from human PBMC removes their ability to manufacture TNFα in response to 100 μM pamidronate (A) and 10 μM risedronate (B). 5×10⁶ PBMC from a healthy donor+/−magnetic depletion of γδ T cells were suspended in 1 ml of R10+/−antigen in 75×5 mm FACS tubes at 37° C. and 5% CO₂ for the times shown. 60 μl of cell supernatant was removed and applied to TNFα ELISA plates in duplicate and developed according to the manufacturers instructions. Standard deviation from the mean of the two replicate ELISAs is shown although, in most cases, this error is smaller than the plot symbol. In (C), exposure to nBPs activates PBMC that express a Vγ9 receptor and not other T cells or B cells. 10⁶ fresh human PBMC were exposed to R10 (top panel) or R10+10 μM risedronate (bottom panel) for 6 hours in an intracellular cytokine staining (ICS) assay. Plots show all the cells in the lymphocyte gate stained for PE-Vγ9 and APC-cytokines (TNFα, IL2, and IFNγ). Exposure to risedronate induces cytokine production only in lymphocytes that express a Vγ9 TCR. The percentage of total lymphocytes in the Vγ9⁺ cytokine⁺ gate shown is indicated in the upper right of each panel. Almost 10% of the lymphocytes expressing a Vγ9 receptor are activated by exposure to nBPs. Similar results were observed with 100 μM pamidronate (data not shown). It is noticeable that exposure to risedronate lowers the expression of the Vγ9 TCR. In (D), intracellular cytokine staining (ICS) shows that nBPs induce direct ex vivo Vγ9-expressing T cells to make TNFα and IL6. Plots are gated to show only Vγ9-expressing lymphocytes. The left hand panels show IL6production induced by 100 μM pamidronate and the right hand panels show TNFα production induced by 10 μM risedronate. The percentage of cytokine positive cells (fluorescence intensity>20) is shown in the upper right corner of each panel.

FIG. 3 shows that statins inhibit nBP-induced TNFα production by human PBMC.

FIG. 3 (A) shows the results of pre-treatment of human PBMC, where 1 μM pravastatin, 100 nM simvastatin or 100 nM fluvastatin, for 2 hours, inhibits their ability to manufacture TNFα in response to 10 μM risedronate. Addition of IPP restores TNFα production and provides a control against any possible toxic effects of these statins. 5×10⁵ cells/well in 200 μl were incubated with nBP for 12 hours at 37° C. and 5% CO₂ in 96 well U-bottomed tissue culture plates. 60 μl of supernatant was applied to a TNFα ELISA plate (Preprotec). Assays were performed in duplicate. Bars show the standard deviation from the mean of two replicate assays.

In FIG. 1 (B), it is shown that pre-treatment of PBMC with statins inhibits nBP-induced activation of Vγ9-expressing T cells, by ICS. Results are shown for three separate experiments. The left panels show results with 1 mM pravastatin; results with 100 nM simvastatin and 100 nM fluvastatin are shown in the middle and right panels respectively. In each case, the experiments show results of 6 hours incubation in R10 only on the top row, R10+10 μM risedronate in row 2, R10+10 μM IPP in row 3, R10+10 μM risedronate+statin in row 4 and R10+10 μM risedronate+statin+10 μM IPP on the bottom row. Statins inhibit nBP-induced activation of Vγ9-expressing T cells (compare rows 2&4). In each case, addition of IPP with the statin (bottom row) rescues activation and controls for any toxic effect of the statins. The percentage of lymphocytes judged to activate in each experiment is shown in the upper right of each panel.

### REFERENCES

1. Russell, R. G., P. I. Croucher, and M. J. Rogers. 1999. Bisphosphonates: pharmacology, mechanisms of action and clinical uses. Osteoporos Int 9 Suppl 2:S66.
2. Adami, S., A. K. Bhalla, R. Dorizzi, F. Montesanti, S. Rosini, G. Salvagno, and V. Lo Cascio. 1987. The acute-phase response after bisphosphonate administration. Calcif Tissue Int 41:326.
3. Gallacher, S. J., S. H. Ralston, U. Patel, and I. T. Boyle. 1989. Side-effects of pamidronate. Lancet 2:42.
4. Schweitzer, D. H., M. Oostendorp-van de Ruit, G. Van der Pluijm, C. W. Lowik, and S. E. Papapoulos. 1995. Interleukin-6 and the acute phase response during treatment of patients with Paget's disease with the nitrogen-containing bisphosphonate dimethylaminohydroxypropylidene bisphosphonate. J Bone Miner Res 10:956.
5. Sauty, A., M. Pecherstorfer, I. Zimmer-Roth, P. Fioroni, L. Juillerat, M. Markert, H. Ludwig, P. Leuenberger, P. Burckhardt, and D. Thiebaud. 1996. Interleukin-6 and tumor necrosis factor alpha levels after bisphosphonates treatment in vitro and in patients with malignancy. Bone 18:133.
6. Thiebaud, D., A. Sauty, P. Burckhardt, P. Leuenberger, L. Sitzler, J. R. Green, A. Kandra, J. Zieschang, and P. Ibarra de Palacios. 1997. An in vitro and in vivo study of cytokines in the acute-phase response associated with bisphosphonates. Calcif Tissue Int 61:386.
7. Kobashigawa, J. A., S. Katznelson, H. Laks, J. A. Johnson, L. Yeatman, X. M. Wang, D. Chia, P. I. Terasaki, A. Sabad, G. A. Cogert, and et al. 1995. Effect of pravastatin on outcomes after cardiac transplantation. N Engl J Med 333:621.
8. Stuve, O., T. Prod'homme, A. Slavin, S. Youssef, S. Dunn, L. Steinman, and S. S. Zamvil. 2003. Statins and their potential targets in multiple sclerosis therapy. Expert Opin Ther Targets 7:613.
9. Stuve, O., S. Youssef, L. Steinman, and S. S. Zamvil. 2003. Statins as potential therapeutic agents in neuroinflammatory disorders. Curr Opin Neurol 16:393.
10 . Marz, W., and W. Koenig. 2003. HMG-CoA reductase inhibition: anti-inflammatory effects beyond lipid lowering? J Cardiovasc Risk 10:169.
11. Crisby, M. 2003. Modulation of the inflammatory process by statins. Drugs Today (Barc) 39:137.
12. Leung, B. P., N. Sattar, A. Crilly, M. Prach, D. W. McCarey, H. Payne, R. Madhok, C. Campbell, J. A. Gracie, F. Y. Liew, and I. B. McInnes. 2003. A novel anti-inflammatory role for simvastatin in inflammatory arthritis. J immunol 170:1524.
13. Blanco-Colio, L. M., J. Tunon, J. L. Martin-Ventura, and J. Egido. 2003. Antiinflammatory and immunomodulatory effects of statins. Kidney Int 63:12.
14. Weitz-Schmidt, G. 2002. Statins as anti-inflammatory agents. Trends Pharmacol Sci 23:482.
15. Shinoda, H., G. Adamek, R. Felix, H. Fleisch, R. Schenk, and P. Hagan. 1983. Structure-activity relationships of various bisphosphonates. Calcif Tissue Int 35:87.
16. Rogers, M. J., D. J. Watts, and R. G. Russell. 1997. Overview of bisphosphonates. Cancer 80:1652.
17. Fleisch, H. 1998. Bisphosphonates: mechanisms of action. Endocr Rev 19:80.
18. Gober, H. J., M. Kistowska, L. Angman, P. Jeno, L. Mori, and G. De Libero. 2003. Human T cell receptor gammadelta cells recognize endogenous mevalonate metabolites in tumor cells. J Exp Med 197:163.
19. Tanaka, Y., C. T. Morita, E. Nieves, M. B. Brenner, and B. R. Bloom. 1995. Natural and synthetic non-peptide antigens recognized by human gamma delta T cells. Nature 375:155.
20. Kunzmann, V., E. Bauer, J. Feurle, F. Weissinger, H. P. Tony, and M. Wilhelm. 2000. Stimulation of gammadelta T cells by aminobisphosphonates and induction of antiplasma cell activity in multiple myeloma. Blood 96:384.
21. Das, H., L. Wang, A. Kamath, and J. F. Bukowski. 2001. Vgamma2Vdelta2 Tcell receptor-mediated recognition of aminobisphosphonates. Blood 98:1616.
22. Kato, Y., Y. Tanaka, F. Miyagawa, S. Yamashita, and N. Minato. 2001. Targeting of tumor cells for human gammadelta T cells by nonpeptide antigens. J Immunol 167:5092.
23. Kato, Y., Y. Tanaka, H. Tanaka, S. Yamashita, and N. Minato. 2003. Requirement of species-specific interactions for the activation of human gamma delta T cells by pamidronate. J Immunol 170:3608.
24. Miyagawa, F., Y. Tanaka, S. Yamashita, B. Mikami, K. Danno, M. Uehara, and N. Minato. 2001. Essential contribution of germline-encoded lysine residues in Jgammal.2 segment to the recognition of nonpeptide antigens by human gammadelta T cells. J Immunol 167:6773.
25. Davodeau, F., M. A. Peyrat, M. M. Hallet, J. Gaschet, I. Houde, R. Vivien, H. Vie, and M. Bonneville. 1993. Close correlation between Daudi and mycobacterial antigen recognition by human gamma delta T cells and expression of V9JPC1 gamma/V2DJC delta-encoded T cell receptors. J Immunol 151:1214.
26. Kunzmann, V., E. Bauer, and M. Wilhelm. 1999. Gamma/delta T-cell stimulation by pamidronate. N Engl J Med 340:737.
27. Gossman, W., and E. Oldfield. 2002. Quantitative structure—activity relations for gammadelta T cell activation by phosphoantigens. J Med Chem 45:4868.
28. Graham, D. Y., H. M. Malaty, and R. Goodgame. 1997. Primary aminobisphosphonates: a new class of gastrotoxic drugs—comparison of alendronate and aspirin. Am J Gastroenterol 92:1322.
29. Lufkin, E. G., R. Argueta, M. D. Whitaker, A. L. Cameron, V. H. Wong, K. S. Egan, W. M. O'Fallon, and B. L. Riggs. 1994. Pamidronate: an unrecognized problem in gastrointestinal tolerability. Osteoporos Int 4:320.
30. Youssef, S., O. Stuve, J. C. Patarroyo, P. J. Ruiz, J. L. Radosevich, E. M. Hur, M. Bravo, D. J. Mitchell, R. A. Sobel, L. Steinman, and S. S. Zamvil. 2002. The HMG-CoA reductase inhibitor, atorvastatin, promotes a Th2 bias and reverses paralysis in central nervous system autoimmune disease. Nature 420:78.
31. Bukowski, J. F., C. T. Morita, and M. B. Brenner. 1999. Human gamma delta T cells recognize alkylamines derived from microbes, edible plants, and tea: implications for innate immunity. Immunity 11:57.

1-19. (canceled)

20. A pharmaceutical formulation comprising an early-stage mevalonate pathway blocker and a bisphosphonate.

21. The pharmaceutical formulation of claim 20, wherein the early-stage mevalonate pathway blocker is an inhibitor of 3-hydroxy-3-methylglutarylcoenzyme A reductase.

22. The pharmaceutical formulation of claim 20, wherein the early-stage mevalonate pathway blocker is a statin.

23. The pharmaceutical formulation of claim 22, wherein the statin is selected from the group consisting of simvastatin, lovastatin, atorvastatin, fluvastatin, mevinolin, and a combination thereof.

24. The pharmaceutical formulation of claim 20 wherein the bisphosphonate is a nitrogen containing bisphosphonate.

25. The pharmaceutical formulation of claim 24, wherein the bisphosphonate is selected from the group consisting of pamidronate, dimethyl pamidronate, alendronate, ibandronate, risedronate, zoledronate, and a combination thereof.

26. The pharmaceutical formulation of claim 20 further comprising a carrier.

27. A method for treating a condition treatable with a bisphosphonate, the bisphosphonate associated with an acute phase response in a patient, the method comprising administering an effective amount of an early-stage mevalonate pathway blocker thereto.

28. The method of claim 27, wherein the patient is human.

29. The method of claim 27, wherein the patient is non-human.

30. The method of claim 29, wherein the non-human patient is selected from the group consisting of a horse and a cat.

31. The method of claim 27 wherein the condition is selected from the group consisting of osteoporosis, Paget's disease, skeletal metastases, fibrous dysplasia, Charcot's arthropathy, sympathetic dystrophy, pachydermic periostosis, aseptic osteomyelitis, multiple myeloma, hypercalcaemia, atherosclerosis, and diabetes.

32. The method of claim 27, wherein the early-stage mevalonate pathway blocker is an inhibitor of 3-hydroxy-3-methylglutarylcoenzyme A reductase.

33. The method of claim 27, wherein the early-stage mevalonate pathway blocker is a statin.

34. The method of claim 33, wherein the statin is selected from the group consisting of simvastatin, lovastatin, atorvastatin, fluvastatin, mevinolin, and a combination thereof.

35. The method of claim 27 wherein the bisphosphonate is a nitrogen containing bisphosphonate.

36. The method of claim 35, wherein the bisphosphonate is selected from the group consisting of pamidronate, dimethyl pamidronate, alendronate, ibandronate, risedronate, zoledronate, and a combination thereof.

37. A kit comprising:
a first medicament comprising an early-stage mevalonate pathway blocker, and
a second medicament comprising bisphosphonate, together with an indication that the first medicament should be taken no later than the second medicament.

38. The kit of claim 37, wherein the indication is that the first medicament should be taken from several hours to several days before taking the second medicament.

39. The kit of claim 37, wherein the medicaments are provided in a form suitable for oral administration.

40. The kit of claim 37, wherein the early-stage mevalonate pathway blocker is an inhibitor of 3-hydroxy-3-methylglutarylcoenzyme A reductase.

41. The kit of claim 37, wherein the early-stage mevalonate pathway blocker is a statin.

42. The kit of claim 41, wherein the statin is selected from the group consisting of simvastatin, lovastatin, atorvastatin, fluvastatin, mevinolin, and a combination thereof.

43. The kit of claim 37 wherein the bisphosphonate is a nitrogen containing bisphosphonate.

44. The kit of claim 43, wherein the bisphosphonate is selected from the group consisting of pamidronate, dimethyl pamidronate, alendronate, ibandronate, risedronate, zoledronate, and a combination thereof.
